(19) United States
(12) Patent Application Publication
(10) Pub. No.: US 2025/0006360 A1
(43) Pub. Date: Jan. 2, 2025

(54) PORTABLE INTEGRATED CONTROLLER FOR REMOTE MEDICAL SUPPORT

(71) Applicant: ELECOM CO., LTD., Changwon-si (KR)

(72) Inventors: Yong Bae JUNG, Changwon-si (KR); Ji Hoon RYU, Changwon-si (KR); Jin Uk KIM, Changwon-si (KR); Keon Sang AHN, Changwon-si (KR); Min Ok JANG, Changwon-si (KR)

(21) Appl. No.: 18/460,148

(22) Filed: Sep. 1, 2023

(30) Foreign Application Priority Data
Jun. 28, 2023 (KR) 10-2023-0083769

(51) Int. Cl. G16H 40/67; G06V 20/50; G10L 15/22; G10L 15/26; G10L 15/30; G16H 50/20; G16H 50/30

(52) U.S. Cl. CPC G16H 40/67; G06V 20/50; G10L 15/22; G10L 15/26; G10L 15/30; G16H 50/20; G16H 50/30; G06V 20/64

(57) ABSTRACT

A portable integrated controller is used in a remote medical support system that includes a visualization device, the portable integrated controller, and a remote medical support server. The portable integrated controller includes a controller unit configured to input data; a display unit configured to display collected and processed information on a screen and provide voice or alarms; a power management unit configured to manage supplied power; a camera unit configured to take pictures or videos; a control unit configured to manage and control individual components in an integrated manner; and a communication unit configured to exchange data. The portable integrated controller runs a recognition and matching engine based on image information shared by the visualization device, and shares generated information with the remote medical support server.

### CROSS-REFERENCE TO RELATED APPLICATION

This application claims priority from Korean Patent Application No. 10-2023-0083769 filed on Jun. 28, 2023, which is incorporated herein by reference in its entirety.

### BACKGROUND

### 1. Technical Field

The present disclosure generally relates to a portable integrated controller used for the rescue of an emergency patient that may occur at a site, and more particularly, to a portable integrated controller for remote medical support, for which a user is equipped with a visualization device at a site, information about the state of a patient is obtained in conjunction with the nearby portable integrated controller and then delivered to a remote medical support server, necessary data is exchanged, and thus smooth rescue is enabled at the site.

### 2. Description of the Related Art

In general, first aid is measures taken immediately to an injured person or an acutely ill person at the site of an accident and is a concept including actions taken to help them recover from injury or disease without medical treatment. Furthermore, in connection with this, communication and a smart device are combined with realistic technology, which is attracting attention in the medical field.

There is emerging interest in the development of a response system that enables a person without specialized training in first aid to perform first aid in an emergency situation in which on-site support is unavailable. Accordingly, there is a demand for a portable integrated controller that can be used in a remote medical support system for immediate initial response at the site at which an emergency patient occurs.

### Related Art Literature

Patent document 1: Korean Patent No. 10-1956972

Patent document 2: Korean Patent Application Publication No. 10-2014-0147135

Patent document 3: Korean Patent Application Publication No. 10-2015-0031173

### SUMMARY

The present disclosure has been conceived to overcome the above-described problems, and an object of the present disclosure is to provide a portable integrated controller for remote medical support that can be used in a remote medical support system for immediate initial response at the site where an emergency patient occurs so that a person without professional training in first aid can perform first aid in an emergency situation in which on-site support is unavailable.

According to an aspect of the present invention, there is provided a portable integrated controller used in a remote medical support system including a visualization device, the portable integrated controller, and a remote medical support server, the portable integrated controller including: a controller unit configured to input data; a display unit configured to display collected and processed information on a screen and provide voice or alarms; a power management unit configured to manage supplied power; a camera unit configured to take pictures or videos; a control unit configured to manage and control individual components in an integrated manner; and a communication unit configured to exchange data; wherein the portable integrated controller runs a recognition and matching engine based on image information shared by the visualization device and shares generated information with the remote medical support server.

The communication unit may include a wired communication module and Wireless Fidelity (Wi-Fi) communication module used for an internal network and a Long-Term Evolution (LTE) communication module used for an external network; and the portable integrated controller may be connected with the visualization device over the internal network and be connected with the remote medical support server over the external network.

The portable integrated controller may further include a simple server unit, and may establish a database capable of managing predetermined emergency action situations through the simple server unit; and the portable integrated controller may check the state of connection with the remote medical support server and allow the simple server unit to support predetermined functions of the remote medical support server when it is determined that the state of connection is unstable.

The portable integrated controller may check the state of connection with the remote medical support server and allow information generated and collected by the simple server unit to be immediately shared with the remote medical support server when it is determined that the state of connection is stable.

The portable integrated controller may further include a voice-to-text conversion unit; and the voice-to-text conversion unit may recognize voice and convert it into text data or recognize text and convert it into voice data, and share the resulting text data or voice data with the remote medical support server.

The portable integrated controller may further include a voice interactive solution unit; and the voice interactive solution unit may have interactive artificial intelligence associated with a medical emergency guide and provide interactive chatbot service while operating in conjunction with the voice-to-text conversion unit.

The portable integrated controller may generate information data on the state of a subject requiring medical support based on data of the voice interactive solution unit, and may share the generated information data with the remote medical support server.

The portable integrated controller may determine the emergency state and risk progress of a subject requiring medical support while operating in conjunction with the remote medical support server, may designate a priority for medical support, and may provide an alarm regarding this.

The portable integrated controller may allow a medical support event to be generated to the visualization device according to the designated priority for medical support.

The portable integrated controller may further include an extension interface unit; the expansion interface unit may include at least one of a communication expansion module, a power management expansion module, an audio expansion module, a camera expansion module, a patient monitoring device extension module, and an Internet of Things (IoT) sensor extension module; and the patient monitoring device expansion module may be connected to a device that measures at least one of heart rate, temperature, electrocardiogram, respiration, oxygen saturation (SpO2), and noninvasive blood pressure (NIBP).

The portable integrated controller may be formed in the shape of a rectangular parallelepiped backpack, and may have a space where the visualization device is accommodated in one side of the inside thereof.

Therefore, the portable integrated controller for remote medical support according to the embodiment of the present disclosure has the advantage of transmitting an on-site situation to the medical support server using the visualization device and high-speed wireless communication and enabling a first responder to receive medical guidance from a medical situation room and offer treatment.

Furthermore, the portable integrated controller has the advantage of offering mixed reality (MR)-based medical response using three-dimensional (3D) space biometric object recognition and matching technology in an emergency situation where on-site support is unavailable.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a conceptual diagram schematically showing a remote medical support system according to an embodiment of the present disclosure;

FIG. 2 is a block diagram briefly showing the configuration of a portable integrated controller according to an embodiment of the present disclosure;

FIG. 3 is a block diagram briefly showing the configurations of a communication unit and an extension interface unit according to an embodiment of the present disclosure; and

FIGS. 4 to 6 are views showing the shape of a portable integrated controller according to an embodiment of the present disclosure.

### DETAILED DESCRIPTION

Hereinafter, the technical spirit of the present invention will be described with reference to the drawings, but this is intended for easier understanding. The scope of the present invention is not limited thereto, and the present invention is only defined based on the scope of the claims.

In the following description of the embodiments of the present disclosure, when it is determined that a detailed description of a known function or configuration may unnecessarily obscure the subject matter of the present invention, the detailed description will be omitted. Like reference numerals designate like components throughout the specification.

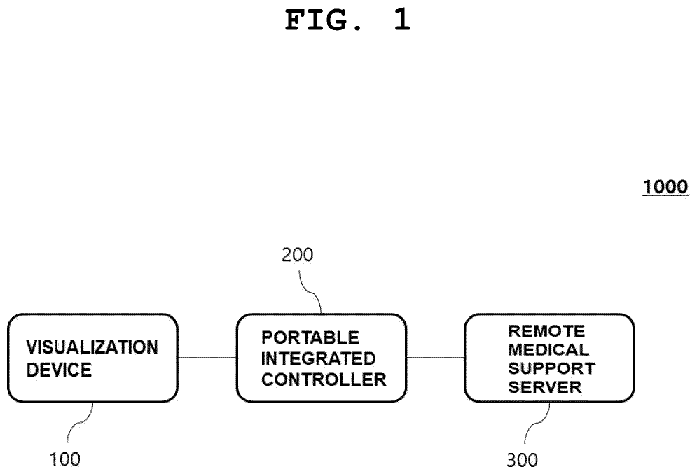

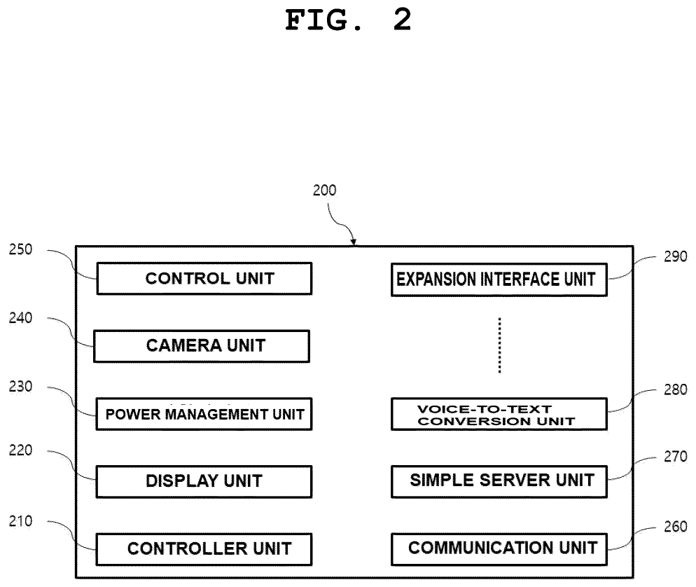

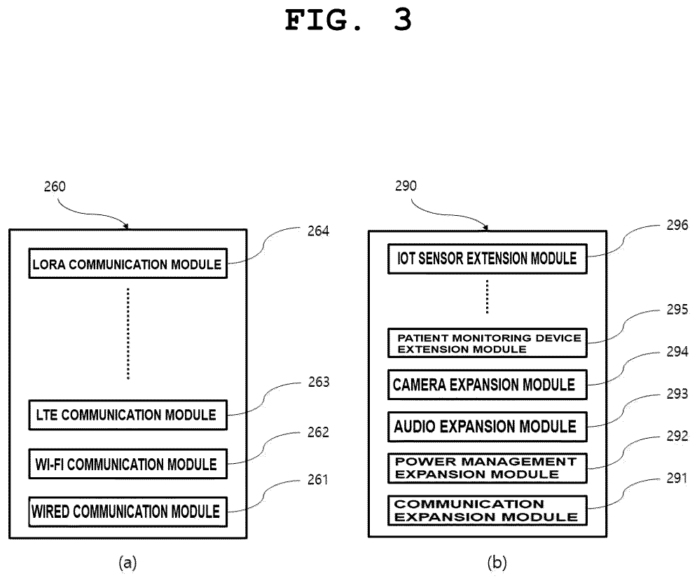

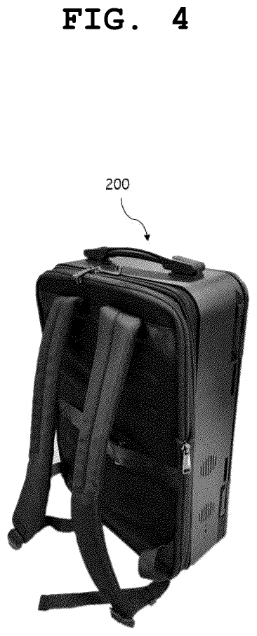

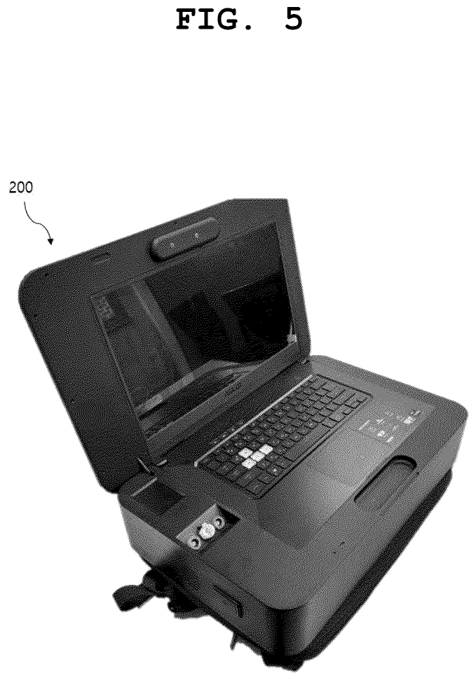

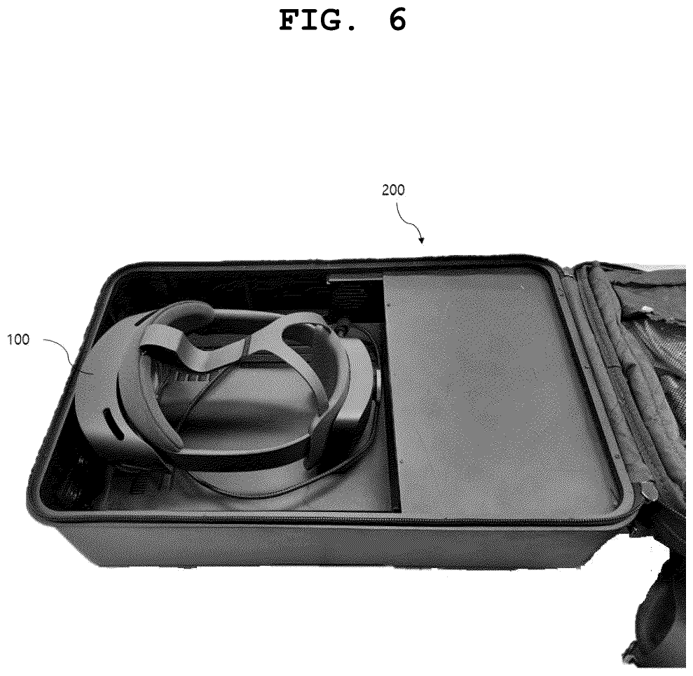

FIG. 1 is a conceptual diagram schematically showing a remote medical support system according to an embodiment of the present disclosure, FIG. 2 is a block diagram briefly showing the configuration of a portable integrated controller according to an embodiment of the present disclosure, FIG. 3 is a block diagram briefly showing the configurations of a communication unit and an extension interface unit according to an embodiment of the present disclosure, and FIGS. 4 to 6 are views showing the shape of a portable integrated controller according to an embodiment of the present disclosure.

### Portable Integrated Controller for Remote Medical Support According to Embodiment of Present Disclosure

Referring to FIGS. 1 to 6, a remote medical support system may include a visualization device 100, a portable integrated controller 200, and a remote medical support server 300. The portable integrated controller 200 for remote medical support according to the embodiment of the present disclosure may be configured to be used in the remote medical support system.

The visualization device 100 is a device capable of implementing augmented reality. The visualization device 100 is connected to the portable integrated controller 200, and is configured to provide information about the state of a patient generated by the portable integrated controller 200, information about a scenario for medical support, and information about a surrounding environment to a user. Although the visualization device 100 preferably includes an MR-based HoloLens, it is not necessarily limited thereto, and may include various types of extended Reality (XR)-based equipment.

A user may perform a contents operation procedure based on a remote medical support scenario through the visualization device 100. A configuration may be made such that by sharing the screen or voice data information of a remote sharing group, smooth medical support is enabled even in a situation without appropriate medical knowledge.

Meanwhile, the visualization device 100 may be configured to share on-site audio and video. Although the vitalization device 100 is preferably configured to enable voice-based manipulation such as voice-based action recognition and voice-based patient history recording, it is not necessarily limited thereto.

Referring to FIG. 2, the portable integrated controller 200 may include a controller unit 210, a display unit 220, a power management unit 230, a camera unit 240, a control unit 250, and a communication unit 260 capable of exchanging data.

The controller unit 210 may be configured to input data inside the portable integrated controller 200.

The display unit 220 may be configured to display collected and processed information on a screen, and the display unit 220 may be configured to provide voice or alarms.

The display unit 220 is preferably configured to display a screen shared by the visualization device 100 or remote medical support server 300 so that the screen can be monitored.

The power management unit 230 may be configured to manage power supplied inside the portable integrated controller 200.

The power management unit 230 may include a detachable and rechargeable battery pack, and may be configured to manage the state of charge of the battery pack.

Furthermore, the power management unit 230 may be configured to detect a connection of an external power source and manage power supplied by the external power source.

The camera unit 240 may be configured to take pictures or videos.

The control unit 250 may be configured to manage and control the individual components of the portable integrated controller 200 in an integrated manner. The control unit 250 may include the function of collecting and diagnosing the state information of each of the components.

The control unit 250 may be configured to receive contents associated with remote medical support from the remote medical support server 300 and run them in the visualization device 100.

In addition, the control unit 250 may include a 3D object recognition and matching engine that is run in the visualization device 100. The control unit 250 may be configured to share information, generated by running the 3D object recognition and matching engine based on image information shared by the visualization device 100, with the remote medical support server 300.

The 3D object recognition and matching engine may be configured to generate real-time 3D information and convert MR-associated information. A markerless method is preferred, but an available method is not necessarily limited thereto.

The 3D object recognition and matching engine may be configured to clearly identify a patient's wound and a treatment target region and generate 3D information that can move a necessary treatment tool to a predetermined position.

The control unit 250 may be configured so that some contents associated with remote medical support are operated in the visualization device 100 by itself through a simple server unit 270 to be described later without relying on the remote medical support server 300.

Meanwhile, the control unit 250 may include an abnormality diagnosis module (not shown) configured to diagnose an abnormal state of the power management unit 230. The abnormal diagnosis module may include: a pattern storage unit configured to learn and store a normal power consumption pattern when the power management unit 230 is in a normal state and an abnormal power consumption pattern when the power management unit 230 is in an abnormal state; a power consumption measurement unit configured to measure the power consumption of the power management unit 230 according to a change in measurement time; a state analysis unit configured to determine whether the power management unit 230 is in a normal state or an abnormal state by comparing the power consumption, measured by the power consumption measurement unit, with the normal power consumption pattern or abnormal power consumption pattern of the pattern storage unit; and an output unit configured to output the results of the power consumption measured by the power consumption measurement unit and the state analyzed by the state analysis unit to the display unit 220.

Referring to FIG. 3(a), the communication unit 260 includes a wired communication module 261 and Wi-Fi communication module 262 used for an internal network and an LTE communication module 263 used for an external network. In this case, the portable integrated controller 200 may configured to be connected to the visualization device 100 over the internal network and to be connected to the remote medical support server 300 over the external network.

Meanwhile, the portable integrated controller 200 may further include the simple server unit 270. The simple server unit 270 may be configured to establish a database capable of managing predetermined emergency action situations by itself.

More specifically, the portable integrated controller 200 may be configured to check the state of connection with the remote medical support server 300 and to, when it is determined that the state of connection is unstable, allow the simple server unit 270 to support predetermined functions of the remote medical support server 300.

In contrast, the portable integrated controller 200 may be configured to check the state of connection with the remote medical support server 300 and to, when it is determined that the state of connection is stable, allow the information generated and collected by the simple server unit 270 to be immediately shared with the remote medical support server 300.

Meanwhile, the portable integrated controller 200 may further include a voice-to-text conversion unit 280. The voice-to-text conversion unit 280 may be configured to recognize voice and convert it into text data or recognize text and convert it into voice data and to share the resulting text data or voice data with the remote medical support server 300. This will enable smooth communication in various situations in the medical field.

Meanwhile, the portable integrated controller 200 may further include a voice interactive solution unit (not shown). The voice interactive solution unit may be configured to have interactive artificial intelligence associated with a medical emergency guide and to provide interactive chatbot service while operating in conjunction with the voice-to-text conversion unit 280. For example, the voice interactive solution unit may be configured to provide a medical guide based on the docent technique.

Furthermore, the portable integrated controller 200 may be configured to generate information data on the state of a subject requiring medical support based on the data of the voice interactive solution unit and to share the generated information data with the remote medical support server 300.

Furthermore, the portable integrated controller 200 may be configured to determine the emergency state and risk progress of a subject requiring medical support while operating in conjunction with the remote medical support server 300, to designate a priority for medical support, and to provide an alarm regarding this.

Moreover, the portable integrated controller 200 may be configured to allow a medical support event to be generated to the visualization device 100 according to the designated priority for medical support.

Meanwhile, the portable integrated controller 200 may further include an extension interface unit 290.

Referring to FIG. 3(b), the expansion interface unit 290 may include at least one of a communication expansion module 291, a power management expansion module 292, an audio expansion module 293, a camera expansion module 294, a patient monitoring device extension module 295, and an IoT sensor extension module 296.

The patient monitoring device expansion module 295 may be configured to be connected to a device that measures at least one of heart rate, temperature, electrocardiogram, respiration, oxygen saturation (SpO₂), and noninvasive blood pressure (NIBP).

The patient monitoring device may be used by the decision of a caregiver in the event of an emergency. Various types of information measured by the patient monitoring device may be shared with the remote medical support server 300 after being collected by the portable integrated controller 200.

For example, the portable integrated controller 200 may be configured to, when a patient monitoring device having electrocardiogramformation is connected for extension, collect and monitor electrocardiogramformation in real time and share the electrocardiogramformation with the visualization device 100 and the remote medical support server 300.

The IoT sensor extension module 296 may be configured to be extended and connected to various IoT sensors capable of collecting surrounding environmental information. Preferably, the IoT sensors include a sensor that collects location information.

The extension interface unit 290 may further include a noise removal module (not shown) configured to remove noise upon extension. The noise removal module may be configured to sample data including noise at predetermined time intervals, to compare the sampled data with the data sampled immediately before that, and to adopt the data sampled later when the difference between the values falls within a predetermined range and adopt the data sampled immediately before that when the difference between the values is out of a predetermined range.

More specifically, in the case where the predetermined range is set to A, when the absolute value of the difference between the sampled data is equal to or smaller than A, the data sampled later may be determined to be noise-free and be adopted. In contrast, when the absolute value of the difference between the sampled data is larger than A, the data sampled later may be determined to contain noise and not be adopted, and the data sampled before may be adopted.

Meanwhile, the sampling time interval and the absolute value of the difference between the sampled data may be changed. Noise may be removed by using a bandpass filter, which is generally used in signal processing.

Referring to FIGS. 4 to 6, the portable integrated controller 200 is preferably formed in the shape of a rectangular parallelepiped backpack for easy portability and has a space where the visualization device 100 is accommodated in one side of the inside thereof, but is not necessarily limited thereto. It is obvious that the portable integrated controller 200 may be configured in various shapes.

The remote medical support server 300 may be connected to the portable integrated controller 200, and may be configured to share information about medical support applicable in an emergency medical situation with the portable integrated controller 200. It is obvious that the remote medical support server 300 may be composed of a single server or a plurality of servers including various servers of related organizations.

The remote medical support server 300 may be configured to periodically update database information including contents associated with medical support and scenario operation procedures, and to share it with the portable integrated controller 200.

Therefore, the portable integrated controller for remote medical support according to the embodiment of the present disclosure has the advantage of transmitting an on-site situation to the medical support server using the visualization device and high-speed wireless communication and enabling a first responder to receive medical guidance from a medical situation room and offer treatment. Furthermore, the portable integrated controller has the advantage of offering MR-based medical response using 3D space biometric object recognition and matching technology in an emergency situation in which on-site support is unavailable.

Meanwhile, although the portable integrated controller for remote medical support according to the embodiment of the present disclosure has been described as being connected to the single visualization device and the single remote medical support server, it is not limited thereto. It is obvious that the portable integrated controller may be connected to a plurality of visualization devices and a plurality of remote medical support servers.

Although the embodiments of the present disclosure have been described with reference to the accompanying drawings, it will be apparent to those skilled in the art that various applications, modifications, and adaptations may be made based on the foregoing description without departing from the scope of the present invention.

What is claimed is:

1. A portable integrated controller used in a remote medical support system including a visualization device, the portable integrated controller, and a remote medical support server, the portable integrated controller comprising:
a controller unit configured to input data;
a display unit configured to display collected and processed information on a screen and provide voice or alarms;
a power management unit configured to manage supplied power;
a camera unit configured to take pictures or videos;
a control unit configured to manage and control individual components in an integrated manner; and
a communication unit configured to exchange data,
wherein the portable integrated controller runs a recognition and matching engine based on image information shared by the visualization device and shares generated information with the remote medical support server.

2. The portable integrated controller of claim 1, wherein the communication unit includes a wired communication module and Wireless Fidelity (Wi-Fi) communication module used for an internal network and a Long-Term Evolution (LTE) communication module used for an external network, and
wherein the portable integrated controller is connected with the visualization device over the internal network and is connected with the remote medical support server over the external network.

3. The portable integrated controller of claim 2, wherein the portable integrated controller further comprises a simple server unit, and establishes a database capable of managing predetermined emergency action situations through the simple server unit, and
wherein the portable integrated controller checks a state of connection with the remote medical support server and allows the simple server unit to support predetermined functions of the remote medical support server when it is determined that the state of connection is unstable.

4. The portable integrated controller of claim 3, wherein the portable integrated controller checks a state of connection with the remote medical support server and allows information generated and collected by the simple server unit to be immediately shared with the remote medical support server when it is determined that the state of connection is stable.

5. The portable integrated controller of claim 1, wherein the portable integrated controller further comprises a voice-to-text conversion unit, and
wherein the voice-to-text conversion unit recognizes voice and converts it into text data or recognizes text and converts it into voice data, and shares the resulting text data or voice data with the remote medical support server.

6. The portable integrated controller of claim 5, wherein the portable integrated controller further comprises a voice interactive solution unit, and
wherein the voice interactive solution unit has interactive artificial intelligence associated with a medical emergency guide and provides interactive chatbot service while operating in conjunction with the voice-to-text conversion unit.

7. The portable integrated controller of claim 6, wherein the portable integrated controller generates information data on a state of a subject requiring medical support based on data of the voice interactive solution unit, and shares the generated information data with the remote medical support server.

8. The portable integrated controller of claim 7, wherein the portable integrated controller determines an emergency state and risk progress of a subject requiring medical support while operating in conjunction with the remote medical support server, designates a priority for medical support, and provides an alarm regarding this.

9. The portable integrated controller of claim 8, wherein the portable integrated controller allows a medical support event to be generated to the visualization device according to the designated priority for medical support.

10. The portable integrated controller of claim 1, wherein the portable integrated controller further comprises an extension interface unit,
wherein the expansion interface unit includes at least one of a communication expansion module, a power management expansion module, an audio expansion module, a camera expansion module, a patient monitoring device extension module, and an Internet of Things (IoT) sensor extension module, and
wherein the patient monitoring device expansion module is connected to a device that measures at least one of heart rate, temperature, electrocardiogram, respiration, oxygen saturation (SpO₂), and noninvasive blood pressure (NIBP).

11. The portable integrated controller of claim 1, wherein the portable integrated controller is formed in a shape of a rectangular parallelepiped backpack, and has a space where the visualization device is accommodated in one side of an inside thereof.
